(19) United States
(12) Patent Application Publication
(10) Pub. No.: US 2023/0393070 A1
(43) Pub. Date: Dec. 7, 2023

(54) METHOD OF DETECTING A BIOFILM IN CLOSED PLANTS

(71) Applicant: WG Holding GmbH, Seewalchen am Altersee (AT)

(72) Inventor: Wolfgang Gauges, Seewalchen am Attersee (AT)

(21) Appl. No.: 18/205,192

(22) Filed: Jun. 2, 2023

(30) Foreign Application Priority Data
Jun. 3, 2022 (AT) A50394/2022

(51) Int. Cl. G01N 21/64

(52) U.S. Cl. CPC G01N 21/6458; G01N 21/6428; G01N 2021/6439

(57) ABSTRACT

A method of detecting a biofilm in closed plants, in which first a rinsing medium is introduced (1) into the emptied plant and then an enzymatic solution for detaching the biofilm is admixed (3) and the mixture is circulated (4) in the plant is described. In a method of the type described above the presence of a biofilm can be reliably detected despite other organic impurities and with simple preparation of the rinsing medium to be measured,—where a sample of the circulated mixture is taken (6) from the plant, then fluorescently labeled (9) with fluorescent markers that specifically bind a biofilm component, and a photometric image of the sample is generated (10), after which a detection signal is output when the characteristic emission wavelengths of those fluorescent markers with which the fluorescently labeled biofilm components present in the sample are labeled are detected in the image.

The invention relates to a method for detecting a biofilm in closed plants, in which first a rinsing medium is introduced into the emptied plant and then an enzymatic solution for detaching the biofilm is admixed and the mixture is circulated in the plant.

Biofilms comprise a mucus layer, are formed by microorganisms in aqueous environments and colonize and contaminate closed plants, such as those used in food production in particular. Since biofilms comprise water-repellent boundary layers and therefore cannot simply be rinsed out of the plant, the cleaning effort for a plant is correspondingly higher. For this reason, it should be determined before cleaning whether biofilm contamination is actually present in order to avoid unnecessary production downtimes and costly cleaning processes.

Methods for the detection of biofilms are known from the state of the art. For example, DE102017005695A1 shows such a method in which a production plant for food manufacturing has a measuring circuit from which a smaller rinsing circuit is branched off. In this rinsing circuit, rinsing water is used as a rinsing medium, to which an enzyme cleaner is admixed for detaching any biofilm that may be present. Then a first TOC (total organic carbon) value is determined for this mixture and the mixture is fed back into the larger measuring circuit. Now the TOC value of the larger measuring circuit is determined as a second TOC value and then compared with the first TOC value. If the second TOC value is higher than the first, a biofilm is detected. If both TOC values are the same, no biofilm is detected.

However, a disadvantage of the prior art is that in order to detect the biofilm, it is assumed that the concentration of the total organically bound carbon in the rinsing water measured at the TOC value originates from a biofilm. However, the measured carbon does not necessarily have to be present as biofilm, as other organic contaminants also influence the TOC value. Furthermore, the volumes of the measuring and rinsing circuit as well as the amount of enzyme cleaner concentration must be determined and two TOC values measured and matched.

The invention is thus based on the task of reliably detecting the presence of a biofilm despite other organic contaminants and with simple preparation of the rinsing medium to be measured.

The invention solves the problem by taking a sample of the circulated mixture from the plant, said sample is fluorescently labeled with fluorescent markers which specifically bind a biofilm component, and a preferably photometric image of the sample is generated, for example by fluorescence microscopy or a photometer, after which a detection signal is output when the characteristic emission wavelengths of those fluorescent markers with which the fluorescently labeled biofilm components present in the sample are labeled are detected in the image. The invention is based on the idea that in order to detect a biofilm, it is not the concentration of organic carbon in the circulated mixture that is determined, but the biofilm components characteristic of the biofilm, for example cells, parts of the DNA of such cells and extracellular three-dimensional structures, such as extracellular polymeric substances (EPS) formed by the cells. By using common fluorescent labeling protocols, the different components of the biofilm can be specifically labeled with fluorescent markers and made detectable and distinguishable by subsequent techniques, such as fluorescence microscopy or photometry. Excess fluorescent markers, or those for which no binding partner is present in the sample, are subsequently washed out, following standard protocols. For example, esterase activity typical of a living cell can be tagged with one fluorescent marker, plasma membrane integrity failure typical of dead cells can be tagged with another fluorescent marker, and EPS can be tagged with yet another fluorescent marker. If different fluorescent markers are used, they can be detected easily and preferably automatically on the basis of their distinguishable, characteristic emission wavelengths in order to distinguish the different biofilm components, for example living cells, dead cells and extracellular three-dimensional structures in the circulated sample. From the measured fluorescence, it can be determined whether a biofilm is present in the sample and subsequently a detection signal can be output, namely when the characteristic emission wavelengths of the fluorescent markers for labeling the characteristic biofilm components are detected. Furthermore, it can be determined, for example, whether the cells that form part of a biofilm that may be present are still alive or already dead, namely when the fluorescent markers for labeling the live or dead cells are detected. Via the measured fluorescence of the different fluorescent markers, the quantitative relationship, i.e. the measured quantum or light yield for each fluorescent marker, between the different biofilm components can already be determined easily and preferably automatically and without calibration. Preferably, the user himself can additionally recognize any three-dimensional structures that may be present and/or the characteristic cell network or other biofilm components in the fluorescence image on the basis of the different fluorescence markers and thereby qualitatively determine whether a biofilm is present. For this purpose, it can, for example, compare the fluorescence image with reference images. Preferably, this qualitative determination is also carried out automatically by neural networks commonly used for image classification. Such neural networks can be trained in advance using images classified by a user. For example, the detection signal, especially in the automated determination, can be output when the light yield of a fluorescent marker to be detected exceeds a predetermined threshold and thus a minimum amount of a biofilm component is detected. Another advantage of the method is that the collection of the circulated sample can be performed as part of a cleaning in place (CIP) procedure for cleaning the equipment and the equipment can be further cleaned after sample collection. The rinsing medium can be rinse water, which is also used as part of a CIP process. Since the enzymatic solution is admixed into the entire rinsing medium present in the system, the correct amount and concentration of enzymatic solution only needs to be done once for a relatively large volume, making it relatively easy to correct measurement inaccuracies in admixture and concentration. A fluorescent marker is any biomolecule that can specifically bind to the desired biofilm component and produce either direct or indirect light emissions in a certain wavelength range. Direct emission in this context means that the fluorescent marker itself emits light in a certain wavelength range after excitation. Indirect emission means that the fluorescent marker interacts with another substance so that this other substance emits light in a certain wavelength range due to this interaction.

In order to make the biofilm detectable not only quantitatively via the light yield, but also qualitatively and thereby preferably reduce the amount of fluorescent markers required, it is proposed that the image of the sample is generated by means of fluorescence microscopy. The enzymatic solution is mixed and dosed according to known stoichiometric methods in such a way that it only detached and does not dissolve the biofilm that may be present. This means that at least a part of the three-dimensional structure of the biofilm is retained and can be detected in the sample by means of fluorescence microscopy. This means that the biofilm can be detected by a user via its three-dimensional structure even if only a relatively small proportion of the labeled biofilm component has been bound to a fluorescent marker. This is because due to the additional information of the spatial distribution of the fluorescent marker obtained during fluorescence microscopy, it is no longer necessary to supersaturate the biofilm component with fluorescent marker using common protocols in order to detect its presence. Here, too, a neural network can be trained on the basis of reference images to recognize the three-dimensional structures of a biofilm and, when recognized, to output the recognition signal.

In order to accelerate enzymatic detaching with simple technical means, the rinsing medium can be tempered to 40-50° C. before admixing the enzymatic solution. The temperature of the rinsing medium provides thermal energy that favors the reaction between biofilm and enzyme and thus facilitates the enzymatic detaching of the biofilm. At the same time, denaturation of the enzymes is avoided at the proposed temperatures.

To determine in advance whether the sample needs to be fluorescently labeled to detect a biofilm, the proportion of organic residues in the sample can be quantitatively determined before the image is generated and no biofilm is detected if the proportion is below a predetermined threshold. This is based on the consideration that exceeding a predefined threshold of organic residues is a prerequisite for the possible presence of a biofilm, as this can only form if sufficient organic residues are present in the sample. Consequently, if a sufficient concentration of organic residues is not measured, it can already be determined that no biofilm can have formed and the procedure can be terminated before the fluorescent labeling is carried out. The threshold can be generated, for example, by taking several measurements of the proportion of organic residue on a sample with biofilm present. The measurement may be, for example, a TOC measurement or a chemical oxygen demand (COD) measurement.

The enzymatic solution may include amylases and/or proteases and/or saccharases, as these enzymes reduce the activation energy to break down the major components of the biofilm.

To promote sufficient detaching of any biofilm present by the enzymatic solution so that it can be reliably detected, the mixture can be circulated in the closed plant at constant temperature for at least one hour before sampling. This provides sufficient time under constant reaction conditions for the enzymatic reaction to proceed, even with relatively small amounts of enzyme in the rinsing medium, in such a way that any biofilm present in the sample is detectable.

The reaction rate of an enzyme also depends on the pH value of the reaction environment. Thus, the maximum reaction rate for different enzymes can occur at different pH values. Therefore, in order to favor the enzymatic reactions even when different enzymes are present in the enzymatic solution, it is suggested that the pH value of the mixture is changed and that mixture with changed pH value circulates in the closed plant for at least one hour before the sample is taken. As a result of these measures, the pH value of the mixture can be adjusted to optimize the reaction rate of one or more of the enzymes present in the enzymatic mixture. In a preferred embodiment of the method, the mixture is first circulated at a first pH value for a first period of time and then circulated at a second pH value for a second period of time to first maximize the reaction rate of a first enzyme and then maximize the reaction rate of a second enzyme. For example, the mixture may first be circulated at an unaffected pH value and then the pH value raised, after which the mixture continues to circulate. For example, the pH value may be raised to 10.

In order to achieve a higher quantum yield when generating the image of the sample and thereby increase the signal-to-noise ratio, it is proposed that the sample is a filter cake obtained by filtration of at least a part of the circulated mixture. Filtration increases the concentration of biofilm components in a given sample volume, thereby providing more binding partners for fluorescent labeling. The resulting increased quantum yield allows more light emitted from the specific fluorescent markers to be detected, facilitating both quantitative and qualitative analysis of the sample.

In order to not only detect the presence of a biofilm, but subsequently to determine pathogens forming the biofilm, it is proposed that a part of the sample is subjected to a polymerase chain reaction process in which a specific deoxyribonucleic acid strand segment is amplified as a biofilm component. As a result of these measures, following standard polymerase chain reaction (PCR) protocols, a section of the pathogens' deoxyribonucleic acid (DNA) can be selected and amplified by PCR. By amplifying the section, if it is present in the sample, it can be detected more easily, allowing the pathogens containing the section to be detected in the sample. Although the use of a PCR method no longer allows precise statements to be made about the original quantity of the section of DNA in the untreated sample, the presence of the section of DNA can however, as a result of these measures, already be detected with small amounts of biofilm components and thus at an early stage. In principle, it does not matter whether the PCR procedure is carried out before, during or after generating the image of the sample. Thus, the determination of the biofilm and the pathogens can be carried out particularly quickly if the image is generated in parallel to the PCR procedure. On the other hand, the determination can be carried out in a more resource-efficient manner if the PCR procedure is only carried out once the presence of a biofilm has been detected by generating the image and the subsequent detection signal.

In order to allow both specific and accurate labeling of the specific biofilm components and to evaluate the image of the sample by technically simple means, it is proposed that the image of the sample is generated as part of an enzyme-linked immunosorbent detection process. In such an enzyme-linked immunosorbent detection process (ELISA), an unlabeled antibody is first bound to a substrate, for example a functionalized surface, and thereby immobilized. This unlabeled antibody has a specific binding site for the biofilm component. Usually, the surface concentration of this antibody varies section by section on the substrate to facilitate a quantitative determination of the biofilm component, so that the following steps can be carried out for several sections. Subsequently, at least part of the sample is added to the bound antibodies so that the biofilm components present in the sample bind specifically to the immobilized antibody. After the following washing step, a detection antibody comprising an enzyme is added, which binds equally specifically but to a different epitope on the biofilm component. Excess detection antibodies are again removed in a further washing step. A detection substrate is then added, which emits light when it undergoes an enzymatic reaction with the enzyme of the detection antibody. Thus, the detection antibody emits light indirectly according to the principle described above. The quantity of the enzymatically reacted and thus light-emitting detection substrate is proportional, among other things, to the quantity of the bound biofilm component and can thus serve to detect it. In this case, the light yield can be determined by means of fluorescence microscopy. In a preferred embodiment, however, a photometer is used, since with this the signal strength can be determined more precisely.

Even when specific antibodies are used, undesired binding and therefore falsification of the measurement result can occur with complex samples such as biofilms, which are composed of a large number of different biomolecules. In order to nevertheless achieve high specificity and thus high measurement accuracy without further pre-treatment steps, it is therefore proposed that the enzyme-linked immunosorbent detection process is a sandwich enzyme-linked immunosorbent detection process. In the sandwich enzyme-linked immunosorbent detection process, the detection antibody does not bind directly to the biofilm component, but to an intermediate antibody bound to the biofilm component. The binding of the intermediate antibody to the biofilm component and the binding of the detection antibody to the intermediate antibody results in an increased overall specificity and a spatial separation of the detection antibody from the biofilm component. As a result, measurement accuracy is increased as m is-binding of the detection antibody can be reduced.

In the drawing, the subject matter of the invention is shown by way of example. The drawing shows in

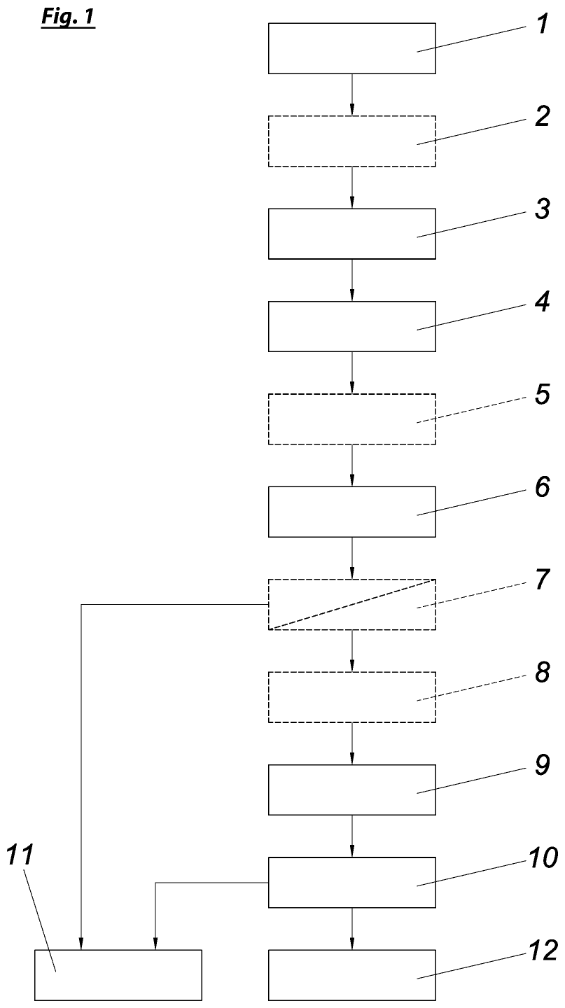

FIG. 1 a schematic representation of the process steps of a method according to the invention, wherein optional process steps are shown in dashed lines.

A method according to the invention for detecting a biofilm in closed plants comprises, in a first method step 1, emptying the plant and subsequently filling it with a rinsing medium. The rinsing medium can preferably be rinse water. This enables, for example, a simple adjustment of the pH value of the rinsing medium and/or a CIP cleaning of the system following the process. Subsequently, in an optional process step 2, the rinsing medium can be tempered to a temperature between 40° C. and 50° C. so that the rinsing medium is already in thermal equilibrium during the subsequent admixing of an enzymatic solution in process step 3 and the reaction rate of the enzymes in the enzymatic solution is accelerated due to the available thermal energy. The admixing of the enzymatic solution in process step 3 can also be carried out sequentially. Thus, several enzymatic solutions can be admixed one after the other. In order to give the enzymes sufficient time to detach any biofilm that may be present, the mixture created by admixing the enzymatic solution to the rinse water is circulated in the closed plant in process step 4. In a preferred embodiment of the process, the mixture is circulated at a constant temperature for at least one hour.

Since different enzymes reach their highest reaction rate at different pH values, the pH value of the mixture can be changed in an optional process step 5 to increase the reaction rate of specific enzymes. Also in this case, the mixture is circulated at the changed pH value for at least one hour to give the enzymes sufficient time to detach any biofilm. In a preferred embodiment, the pH value of the mixture may be changed several times to achieve the optimal reaction rates of different enzymes in succession. The enzymatic solution is composed in such a way that the biofilm is detached by the enzyme activity, i.e. parts of the biofilm pass into the circulated mixture without the three-dimensional structures characteristic of the biofilm being chemically dissolved.

In process step 6, a sample of the circulated mixture is taken, which contains parts of the biofilm due to the enzyme activity described above, if this is present in the system. In an optional process step 7, the proportion of organic residue can already be determined quantitatively on the basis of a sample. For this purpose, a TOC or a COD method can be used, for example, and its result can be compared with a predefined threshold. If it can already be determined in this optional process step that there are not enough organic substances to form a biofilm in the circulated mixture, the absence of a biofilm can already be determined and the process can be terminated at this point in process step 11, outputting the absence of a biofilm. However, if enough organic substances are detected to form a biofilm, the procedure can be continued with the optional procedure step 8 or immediately with procedure step 9. Alternatively or additionally, part of the sample is subjected to a PCR procedure in optional step 7, in which a section of the deoxyribonucleic acid of a pathogen contained in the biofilm is selected and replicated by the PCR procedure. The quantity of the section obtained at the end of the PCR procedure can be used to determine the presence of pathogens comprising this section.

In optional step 8, the sample can be filtered to form a filter cake in order to increase the proportion of the substances to be detected in the sample volume.

The sample is then fluorescent marked in step 9 using standard protocols. In the embodiment described here, directly emitting fluorescent markers are used, whereby the different components of the biofilm to be detected are specifically labeled with fluorescent markers of characteristic emission wavelengths and subsequently detected via fluorescence microscopy. Alternatively, indirectly emitting fluorescent markers can be used, for example, which convert detection substrate in an enzymatic reaction so that the detection substrate emits light. In method step 10, an image of the sample is generated and evaluated. Preferably, different fluorescent markers can be used for this purpose, which can be easily distinguished due to the different emission wavelengths. The image can be evaluated quantitatively. This means that for each fluorescent marker the photonic yield can be quantified and thereby the presence of the different fluorescent marker-bound components of the biofilm can be detected. For this purpose, for example, the quantified photonic yield for a fluorescent marker can be compared with a predetermined reference value and a detection signal can be output if the yield exceeds the reference value. Here, it is advisable to compare the values measured on the sample with a reference image that has been labeled and washed according to the same protocol as the sample, but does not contain binding partners for the fluorescent labels. Furthermore, a ratio of the different components of the biofilm can be estimated easily and without calibration from these measured values. The image can also be examined qualitatively, as the fluorescently labeled cells and the fluorescently labeled three-dimensional structure are recognizable on the image. Again, it is advisable to compare the image with the reference image of a biofilm, which can be done either by the user or by an automated comparison algorithm. As an alternative to fluorescence microscopy, an enzyme-linked immunosorbent detection process (ELISA) can be performed, for example, preferably using indirectly emitting fluorescent markers. If, according to the evaluation, it is determined that no biofilm is present, this is output in process step 11. If a biofilm is detected, a detection signal is output in method step 12.

1. A method of detecting a biofilm in a closed plant, said method comprising:
introducing a rinsing medium into the plant when emptied; and then
admixing an enzymatic solution that detaches the biofilm and circulating a mixture therefrom in the plant;
taking a sample of the circulated mixture from the plant; then
fluorescently labeling the sample with one or more fluorescent markers that each specifically bind to a respective biofilm component; and
generating a photometric image of the sample; and then
outputting a detection signal when there is detection in the image of one or more characteristic emission wavelengths of any of said one or more of said fluorescent markers that have fluorescently labeled any of the biofilm components present in the sample.

2. The method according to claim 1, wherein the image of the sample is generated using fluorescence microscopy.

3. The method according to claim 1, wherein the rinsing medium is tempered to 40-50° C. before admixing the enzymatic solution.

4. The method according to claim 1, wherein a proportion of organic residues in the sample is quantitatively determined before the image is generated and no detection signal is output if the proportion is below a predetermined threshold.

5. The method according to claim 1, wherein the enzymatic solution comprises one or more enzymes from the group consisting of amylases, proteases, and saccharases.

6. The method according to claim 1, wherein the mixture is circulated in the closed plant at constant temperature for at least one hour before the taking of the sample.

7. The method according to claim 1, wherein a pH value of the mixture is changed and said mixture with the changed pH value circulates in the closed plant for at least one hour before the taking of the sample.

8. The method according to claim 1, wherein the sample is a filter cake obtained by filtration of at least a part of the circulated mixture.

9. The method according to claim 1, wherein a part of the sample is subjected to a polymerase chain reaction process in which a specific deoxyribonucleic acid strand segment is amplified as a biofilm component.

10. The method according to claim 1, wherein the image of the sample is generated as part of an enzyme-linked immunosorbent detection process.

11. The method according to claim 10, wherein the enzyme-linked immunosorbent detection process is a sandwich enzyme-linked immunosorbent detection process.

12. The method according to claim 2, wherein the rinsing medium is tempered to before admixing the enzymatic solution.

13. The method according to claim 2, wherein a proportion of organic residues in the sample is quantitatively determined before the image is generated and no detection signal is output if the proportion is below a predetermined threshold.

14. The method according to claim 2, wherein the enzymatic solution comprises one or more enzymes from the group consisting of amylases, proteases, and saccharases.

15. The method according to claim 14, wherein the mixture is circulated in the closed plant at constant temperature for at least one hour before the taking of the sample.

16. The method according to claim 14, wherein a pH value of the mixture is changed and said mixture with the changed pH value circulates in the closed plant for at least one hour before the taking of the sample.

17. The method according to claim 4, wherein the sample is a filter cake obtained by filtration of at least a part of the circulated mixture.

18. The method according to claim 14, wherein a part of the sample is subjected to a polymerase chain reaction process in which a specific deoxyribonucleic acid strand segment is amplified as a biofilm component.

19. The method according to claim 9, wherein the image of the sample is generated as part of an enzyme-linked immunosorbent detection process.

20. The method according to claim 19, wherein the enzyme-linked immunosorbent detection process is a sandwich enzyme-linked immunosorbent detection process.
